(19) United States
(12) Patent Application Publication
(10) Pub. No.: US 2009/0246126 A1
(43) Pub. Date: Oct. 1, 2009

(54) Thulium-based capsule and devices for use in high dose rate brachytherapy

(75) Inventor: Gad Shani, Omer (IL)

Correspondence Address: NIXON & VANDERHYE, PC, 901 NORTH GLEBE ROAD, 11TH FLOOR, ARLINGTON, VA, 22203, US

(73) Assignee: Ben-Gurion University of the Negev Research and Development Authority, Beer-sheva (IL)

(21) Appl. No.: 12/385,216

(22) Filed: Apr. 1, 2009

Related U.S. Application Data
(63) Continuation-in-part of application No. PCT/IL2007/001196, filed on Oct. 7, 2007.

(51) Int. Cl. A61K 51/12; A61K 51/00

(52) U.S. Cl. 424/1.29; 424/1.61

(57) ABSTRACT

A capsule for high dose rate brachytherapy, wherein the capsule comprises within its interior space thulium-170, and further comprises at least one layer of a radiation emission modifying metal (e.g., gold), wherein said layer is provided either internally within the capsule or on the outer surface thereof.

Brachytherapy is a treatment method in which at least one ionizing radiation source is inserted into, or adjacent to, a diseased site, in order to treat the disease. There are two common Brachytherapy treatment types: i) High Dose Rate (HDR); and ii) Low Dose Rate (LDR), which are distinct from one another by the intensity of the radiation source, its form and its method of use. HDR is typically defined as a dose rate of at least 12 Gy/h delivered to the prescription point(s).

In HDR brachytherapy, a high intensity (about 3 to 30 curie) radiation source is used and it is typically inserted into the tumor for a short period of time, typically less than 30 minutes per fraction. HDR brachytherapy is currently applied in treatments of prostate, breast, cervix, uterus, lung, eye and other anatomical sites.

From a radiation safety standpoint, a major advantage of HDR brachytherapy compared with LDR (which usually entails the permanent implantation of radioactive material) is the fact that the patient leaves the hospital without any implanted radioactive material. All of the radioactive material used in the treatment is completely retracted from the patient at the conclusion of the treatment session.

In HDR brachytherapy the radiation source is generally stored in a lead (or other high-density, high-Z material) shielding, and attached to one end of a flexible steel wire. The other end of the wire is attached to a system used for introducing the radiation source into the required irradiation location in the body of the treated subject, typically through a guiding catheter. The positioning of the radiation source in HDR brachytherapy is more accurate than in LDR brachytherapy, wherein the implanted radiation sources can migrate during the time period in which the radiation sources are in the body of the treated subject. Therefore, in HDR the irradiation time is very well controlled, and the dose of radiation delivered to the patient and the distribution of the radiation in the body of the patient is relatively accurate. In HDR, the dose distribution can also be more finely controlled than is possible with LDR. The source dwell time at each dwell position in the implant can be controlled. In permanent-implant LDR, all of the radioactive sources remain in the patient in perpetuity, precluding such an opportunity for dose optimization.

For the aforementioned reasons, HDR brachytherapy is becoming a popular treatment. The most commonly used radionuclide for HDR brachytherapy is ¹⁹²Ir. ¹⁹²Ir emits a large number of gamma rays photons having energy levels in the range of 66 keV (x-rays) to 885 keV. The highest intensity in the ¹⁹²Ir photon spectrum is at the energy level of 317 keV, the next intense energy level is 468 keV. Photons emitted in these energy levels are highly penetrating in tissue and reach much beyond the tumor limits.

The HDR device described in US 2006/135841 comprises a safe for storing a capsule comprising a radioactive source and a transport wire wound onto a reel and connected to the capsule for delivering it via a transport tube to an applicator which has been introduced into a tissue to be irradiated. The radiation source in this device may be prepared from Ytterbium-169, Iodine-125, Palladium-103, Thulium-170 or Tungsten-181.

A radioactive radiation source is described in U.S. Pat. No. 5,282,781, wherein the radiation source is composed of a cylindrical tube having in a proximal section thereof a backbone wire for enhancing the flexibility of the tube and a cylindrical Ir-192 radioactive source placed adjacent to said backbone wire, wherein the cylindrical tube is sealed by a cylindrical plug, and wherein the external surface of the cylindrical tube is plated with gold (or other non-oxidizing material) throughout its length to preclude oxidation of the plated surface and to improve the sealing of the tip.

A flexible radiation source is described in U.S. Pat. No. 6,442,822, said radiation source, which is capable of being maneuvered through various body passages, comprise a thin cylindrical flexible tube having a modified segment at its proximal end and an unmodified section at its distal end, said sections are separated by a sealing plug, wherein the modified section contains a radioactive core made of various isotopes, and its wall thickness is thinner than the wall thickness of the unmodified section. The radiation source is optionally coated with a non-oxidizing agent, such as gold.

The inventor has now found that a beta-gamma radiation source, and more specifically, the isotope thulium 170 (¹⁷⁰Tm), may be effectively used as a radiation source for high dose rate brachytherapy, in combination with a radiation emission modifying metal, which is preferably selected from the group consisting of gold (Au), platinum (Pt) and cerium (Ce). It has been found that the radiation emitted from thulium 170, which is composed of beta radiation (electrons) and gamma radiation (photons), may be favorably modified by coating the thulium 170 with one or more of the metals listed above, and more specifically with gold or cerium. By appropriately adjusting the thickness of the metal coating onto the thulium, it is possible control the intensities of the beta and gamma radiations emitted therefrom, generating a ¹⁷⁰Tm based high dose rate brachytherapy radiation source, characterized in that (i) the level of beta radiation emitted therefrom is reduced in comparison with that generated by ¹⁷⁰Tm in the absence of a radiation emission modifying metal; and further characterized in one or more of the following features: (ii) the photon emission spectrum of said source exhibits one or more peaks in the region between 30 keV and 84 keV energy levels, or between 65 and 84 keV, indicative of photons emitted by the layer(s) of the radiation emission modifying metal; (iii) the photon emission spectrum of said source comprises in addition to the discrete peaks also a continuous region (below 20 keV), indicative of the emission of braking energy associated with the layer(s) of the radiation emission modifying metal; and (iv) the photon emission spectrum of said source displays a lower ratio between the intensities of the characteristic peaks at 84 keV and 52 keV energy levels, relative to the ratio observed in the corresponding spectrum of ¹⁷⁰Tm (in the absence of a radiation emission modifying metal).

Photon spectrum measurements may be carried out using a conventional gamma spectrometry system, which includes: a gamma solid state detector, either HPGe, Si(Li) or CdZnTe (CZT), a high voltage supply, preamplifier, amplifier and a multichannel analyzer. Changes in the emission of beta radiation can be determined by applying a radiation detector, either a thin window ion chamber or a scintillator.

The high dose rate brachytherapy radiation source provided by the invention allows utilizing a considerable portion of the beta emission generated by thulium-170, while reducing the intensity of the gamma radiation of thulium-170, especially, the 84 keV ray of thulium-170. It should be noted that the greater the energy of the photon, the greater its traveling distance is and hence its probability of interacting and damaging healthy tissues which are far from the tumor to be treated. More specifically, by converting a portion of the gamma rays emitted from the thulium-170 to low energy photons generated by the radiation emission modifying metal, new low energy photons are produced, which have higher probabilities of interacting with a relatively small diseased volume (the tumor), without damaging adjacent healthy tissues, as compared to the 84 keV ray of ¹⁷⁰Tm.

Accordingly, the present invention primarily relates to a capsule for high dose rate brachytherapy, wherein the capsule comprises within its interior space thulium-170, and further comprises at least one layer of a radiation emission modifying metal, wherein said at least one layer is provided internally within the capsule, or on the outer surface thereof, or both.

The activity of the radiation source provided by the invention is preferably not less than 3 curie, e.g., between 3 and 70 curie, more preferably between 3 and 50 curie (e.g., 3-30 curie). The term “capsule”, as used herein, refers to a thin casing suitable for enclosing a radioactive source. The casing is deliverable to the diseased site through a catheter, by means of a guidewire, using an afterloader, in accordance with high dose rate brachytherapy protocols. Particularly preferred is a titanium capsule. Capsules of various structures may be used according to the present invention for incorporating the thulium therein, such as those described, for example, in U.S. Pat. No. 6,179,768, U.S. Pat. No. 6,196,964 and U.S. Pat. No. 7,077,800. These references also describe combinations of a capsule and a guidewire, in order to allow the delivery of the capsule to the diseased site (the tumor). The metal of which the capsule is made is of course distinct from the radiation emission modifying metal to be applied in accordance with the invention.

The thulium 170-containing capsule provided by the present invention preferably comprises a gold, platinum or cerium coating which at least partially, and preferably entirely, surrounds the thulium 170, said coating being either interposed between the radioactive isotope and the walls of the capsule, or alternatively, applied onto the outer faces of the capsule. The capsule is capable of being delivered into a body site through a catheter, and preferably has an activity in the range of 3-50 curie, more specifically in the range of 3-30 curie.

The capsule described above may be produced, for example, by coating thulium-169 with one or more layers of a radiation emission modifying metal (gold), placing the coated thulium-169 in a capsule, e.g., titanium capsule, sealing the capsule and submitting the same to a neutron activation in order to convert the thulium-169 into thulium-170. Alternatively, the coating is applied onto the capsule's outer faces. It is noted that thulium-169 to be converted according to the present invention to the radioactive thulium-170 is available in the form of a cylindrical body, namely, a wire of small diameter This simplifies the preparation of the capsule and devices of the invention, as the shape of commercially available thulium 169 readily fits into the capsule. The activation parameters for transforming the natural isotope thulium-169 into thulium-170 are set forth below.

The metal coating, specifically the gold coating, is applied directly onto the thulium, or onto the outer surface of the casing, by means of various techniques. The thickness of the radiation emission modifying metal coating is preferably between a few atomic layers and up to 0.15 mm, and more preferably in the range between 0.02 and 0.1 mm. The thickness of the coating may be specifically tailored in order to adjust the radiation profile of the capsule (namely, to determine the proportions of the beta and gamma radiation generated by the capsule). As illustrated herein below, a gold layer of thickness 10-50 micron has been found effective for turning a portion of the beta energy emitted by thulium 170 into photons energy.

One possible technique for forming the radiation emission modifying metal coating is by means of electroplating, wherein the thulium is immersed in a solution containing gold ions and an electric current is passed through the solution to deposit a gold coating onto a thulium piece. Electroplating conditions (suitable gold solutions, medium stirring rates and current densities required for obtaining desired gold coatings) are known in the art. A second method is by gold evaporation. Heat is applied to a gold wire or rod (generally by means of electric current). Due to the high temperature, gold atoms are evaporated and deposited on the thulium.

A further technique for making the gold coating is known as “spattering”, and has been found to be especially useful for forming relatively thin gold coatings onto the thulium (from 10 nm and up to 1 μm). A gold electrode and the thulium piece to be coated are positioned in a closed chamber and are separated by a distance in the range of 3 and 8 cm. The chamber is maintained under argon atmosphere at a low, constant pressure. A voltage of the order of 1-2 kV is applied between a gold electrode (directly connected to a power source) and the thulium piece to be coated. The electric field generated in the chamber ionizes the gas and causes a current to flow into the gold electrode. This results in gold being spattered from the electrode and being deposited onto the thulium piece. The thickness of the gold coating thus formed onto the thulium is given by the following equation:

Gold Thickness [Å]=k·I·t

Where I is the current in mA, t is the spattering time in minutes and k is a constant characteristic of the device employed for the spattering.

The simplest technique for forming a gold or cerium coating is by wrapping the thulium piece with one or more gold or cerium foils of a desired thickness. Thin gold and cerium foils of various thicknesses are commercially available (Goodfellow, England).

According to a preferred embodiment of the invention, the radiation emission modifying metal completely surrounds the thulium and has a substantially uniform thickness. By the term “substantially uniform thickness” it is meant that the tolerance in the thickness of the coating is not more than ±10%. Keeping the thickness within the said tolerance will reduce the source anisotropy component resulting from variations in coating thickness. The intensity of the isotropic beta radiation generated by the source is more than 10%, and preferably in the range between 20% and 70%, and more preferably in the range between 20 and 50%, relative to the intensity of the beta radiation emitted by a corresponding non-coated thulium 170. Changes in the emission of beta radiation can be determined by applying a radiation detector, either a thin window ion chamber or a scintillator.

The invention also provides a high dose rate brachytherapy device, which comprises as a radiation source thulium-170 and at least one layer of a radiation emission modifying metal surrounding (at least partially, and preferably entirely) said thulium-170, wherein the photon emission spectrum of the source exhibits one or more of the characteristics set forth above (peaks in the region between 30 keV and 84 keV energy levels, or between 65 keV and 84 keV, and low energy continuum spectrum at below 20 keV, indicative of photons emitted by the layer of the radiation emission modifying metal).

The high dose rate brachytherapy device of the invention preferably comprises thulium-170 cylinder surrounded (in a coaxial fashion, and also on its bases) with a radiation emission modifying metal. Thus, the device relates to a thin, flexible (stainless steel) guiding wire, having a distal end and a proximal end, with thulium-170 segment being attached to said distal end, wherein the thulium-170 segment is at least partially coated by a radiation emission modifying metal as described above. More specifically, the present invention relates to a high dose rate brachytherapy device, comprising:

a) a thulium-170 radioactive section attached to one end of a guiding wire, said radioactive section and a portion of said guiding wire being placed within the interior of a shell and affixed to the walls thereof, with the proximal end of the guiding wire extending beyond said shell; and
b) a radiation emission modifying metal, provided either internally within the shell or on the outer surface thereof.

Preferably, the radiation emission modifying metal, and more specifically gold or cerium, is provided as a thin coating onto the surface of said radioactive section, such that said gold or cerium layer is interposed between said radioactive section and said shell. The thickness of the gold layer may be up to 0.15 mm. Alternatively, a gold (or cerium) layer is externally applied onto the shell.

The distal end of the device, to be positioned in proximate with the body site to be irradiated, is defined by said shell and the thulium isotope placed therein, attached to one end of the guiding wire; The proximal end of the device is defined by the second end of said guiding wire to be connected to a suitable machinery, as will be described in more detail below.

More specifically, the device has an essentially cylindrical symmetry. The shell may be provided in the form of a cylindrical surface having one open base. The thulium 170 isotope, in the form of a cylindrical body, is confined within said shell, with the guiding cable extending outwardly through the open base of said shell. Alternatively, the shell may be a tube, e.g., commercially available titanium tube, such that its distal end needs to be sealed following the insertion of the isotope therein.

In another aspect, the present invention provides a high dose rate brachytherapy method, which comprises placing proximate to a diseased site (e.g., tumor site) at least one radiation source comprising thulium 170 provided with a coating of a radiation emission modifying metal as described above, which metal is preferably gold or cerium, wherein the activity of said radiation source is not less than 3 curie, and preferably in the range between 3 and 70 curie, and delivering radiation dose to said diseased site for a period of time, typically less than 30 minutes per fraction. A typical radiation dose can be in the range between 5 and 10 Gy (Gray) per fraction.

### BRIEF DESCRIPTION OF THE DRAWINGS

The present invention is illustrated by way of example in the accompanying drawings, in which similar references consistently indicate similar elements and in which;

FIGS. 1a and 1b illustrate a longitudinal-section view of a general structure of a HDR brachytherapy device of the invention;

FIG. 2 shows a longitudinal-section view of a HDR brachytherapy device of the invention wherein the radioactive section is enclosed in a tube;

FIG. 3 shows a longitudinal-section view of a brachytherapy device of the invention, wherein the heavy metal coating is applied externally;

FIG. 4a shows a longitudinal section view of a capsule of the invention encased in a tubular body and FIG. 4b shows a cross-sectional view of the capsule.

FIG. 5 shows the characteristic photon spectrum emitted by ¹⁷⁰Tm;

FIG. 6 shows a photon energy spectrum measured for a ¹⁷⁰Tm radiation source of the invention having a 0.1 mm gold layer coating;

FIG. 7 shows a photon spectrum of a ¹⁷⁰Tm radiation source in which the radiating core was encased in a titanium capsule having wall thickness of about 0.05 mm; and

FIG. 8 schematically illustrates a delivery system for introducing a radiation source into an irradiation site in a treated subject.

FIG. 9 shows a photon energy spectrum measured by CZT detector for a ¹⁷⁰Tm radiation source of the invention having a 0.2 mm gold layer coating

FIG. 10 is a graph in which the fraction of the thulium-170 beta energy, which is converted into photons, is plotted as a function of the thickness of the gold coating.

It should be noted that the embodiments exemplified in the Figures are not intended to be in scale and are in diagram form to facilitate ease of understanding and description.

### DETAILED DESCRIPTION OF PREFERRED EMBODIMENTS

The present invention provides a radiation source, which may be used for high dose rate brachytherapy. The beta-gamma radiation source of the invention comprises the isotope thulium-170 (¹⁷⁰Tm), wherein a preferable ratio between the beta and the gamma radiations emitted from said isotope is obtained by coating the radiation source by one or more layers made of metals that are most preferably selected from the group consisting of gold, platinum and cerium.

The ¹⁷⁰Tm radiation source is preferably obtained by neutron activation of natural thulium-¹⁶⁹Tm. The activated ¹⁷⁰Tm emits both beta and gamma rays and therefore it can be used for treatment of small tumors as well as large tumors. The beta rays may be used for treating small tumors, while the gamma photons are more suitable for treating large tumors.

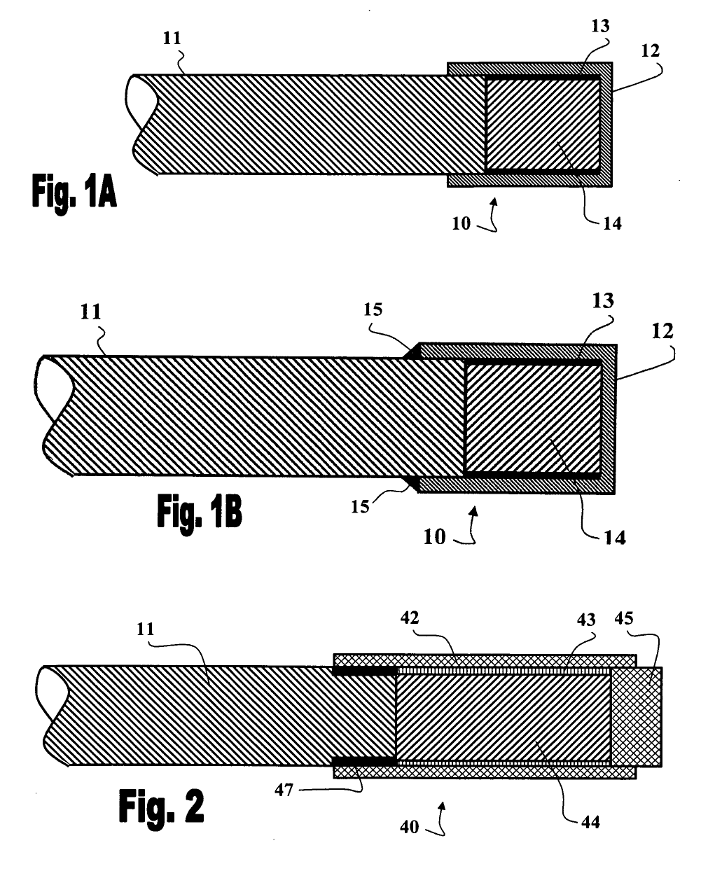

With reference to FIG. 1a, a brachytherapy device 10 of the invention comprises a radioactive section 14 made of thulium-170, and having a radiation emission modifying metal (e.g., Au, Pt or Ce) coating 13 surrounding the same (the radioactive isotope is produced by activation of thulium-169 under conditions that are described hereinafter). The radioactive ¹⁷⁰Tm section 14 is preferably a cylindrical body having a diameter generally in the range of 0.6 to 1.0 mm, preferably about 1 mm (or a little smaller), and having a length which corresponds to the desired activity and is typically less than 1 cm. For example, possible lengths of the radioactive core 14 are 1.5, 3 or 5 mm, for providing activities of 4 curie, 8 curie, or 15 to 30 curie, respectively. These preferable lengths give volumes of about 1.1775, 2.355 and 3.925 mm³, respectively, and weight of 10.95, 21.98 and 36.5 mg, respectively. The number of ¹⁶⁹Tm atoms present in the pre-irradiated source is of importance since the number of ¹⁷⁰Tm atoms produced by the neutron activation is relative to the number of ¹⁶⁹Tm atoms being activated. The numbers of activated Tm169 atoms are about: 3.89×10²², 7.78×10²² and 1.29×10²³, respectively. The number of ¹⁷⁰Tm atoms obtained in the neutron activation equals the product of the number of ¹⁶⁹Tm atoms irradiated by the neutron fluence multiplied by the activation cross-section. (Neutron fluence=neutron flux times activation time). The activity (number of disintegrations per second) of the ¹⁷⁰Tm core 14 is given by the number of ¹⁷⁰Tm atoms times the decay constant.

The ¹⁷⁰Tm 14 is preferably contained in a cap 12 having an outer diameter of about 1.1 mm and wall thickness of about 0.05 mm. A thin wall is important for the utilization of the beta rays. A thicker wall will absorb a certain percent of the beta rays. The length of cap 12 depends on the length of the Tm section 14. In a preferred embodiment of the invention, cap 12 is made from a type of inert metal, such as, but not limited to titanium or stainless steel, preferably from titanium, and the length of cap 12 is preferably about 1 mm longer than the length of core 14.

The device is prepared as follows. A piece of thulium-169, coated with gold is inserted into the cap 12. Gold coating may be accomplished by numerous conventional methods. For example, electroplating may be applied, wherein the thulium is immersed in a gold solution and an electric current is passed through the solution to form a gold coating onto the object. A second method is by gold evaporation. Heat is applied to a gold wire or rod (generally by electric current). Due to the high temperature gold atoms are evaporated and deposited on the thulium A third way is to wrap the thulium with a gold foil of the desired thickness. Thin gold foils of various thicknesses are commercially available (Goodfellow, England). The thulium section 14 may be attached to cap 12, for example, by glue or by being snugly pressed thereinto.

The titanium cap 12 with the natural thulium (¹⁶⁹Tm-before activation) coated by one or more heavy metal layers 13 contained thereinside, is transferred for neutron activation to a nuclear reactor.

Preferably, the thulium is activated by a neutron flux on the order of 10¹⁵ n/cm² s, such that the activation time required is relatively short (e.g., 7-15 days). The activation time should be sufficient to obtain the desired activity, as explained above.

After obtaining the required activity, a guiding wire 11 is inserted into cap 12. Guiding wire 11 is preferably made from a stiff and flexible material, such as, for example, steel. The length of wire 11 may generally be in the range of 200 to 2000 mm, preferably about closer to 2000 mm, and its diameter may generally be in the range of 0.7 to 10 mm, preferably about 1.0 mm.

The attachment of guiding wire 11 (to the titanium cap 12 comprising the thulium core 14 coated by gold layer 13) may be carried out by means of welding, or an epoxy glue under pressure (the cap volume between the thulium and the steel rod is filled with epoxy). The diameter of guiding wire 11 is preferably slightly smaller than the inner diameter of cap 12, such that a distal end portion of guiding wire 11 may be snugly introduced thereinto until it abuts core 14. In this way, wire 11 may be pressed into cap 12, and after the epoxy glue is dried a well-sealed strong connection is obtained.

The device 10 with the guiding wire 11 is preferably kept in an appropriate shield (e.g., 97 in FIG. 8), and delivered through a properly placed guiding catheter towards the treatment site (diseased tissue, e.g., cancerous tumor) within the body of the treated subject, by means of a remotely operated mechanism (possible delivery system is described hereinbelow with reference to FIG. 8). The device 10 is maintained in the treatment site within said guiding catheter (in the tumor) for a predetermined time (e.g., ranging between 10-30 minutes), sufficient to deliver the required dose of radiation (e.g., 5-10 Gy/fraction). The selected dosage regimen will depend on the type of pathology being treated and other factors associated with the patient.

FIG. 1b illustrates a brachytherapy device similar to the one shown in FIG. 1a, wherein the cap 12 is fixed to the guiding wire 11 by means of welding 15.

FIG. 2 shows a longitudinal-section view of another preferred embodiment of a device 40 of the invention, wherein the radiating core 44 is enclosed in a tube 42. The production of radiation source 40 may be substantially similar to that of device 10 shown in FIG. 1. Similarly, a cylindrical Tm core (Tm wire) is covered by one or more layers of metal coatings 43 (e.g., Au, Pt or Ce), and it is then snugly introduced into tube 42 via one of its openings. The metal coatings 43 may be applied by means of conventional metal plating techniques, as described above, or alternatively, by wrapping the Tm core 44 by a metal foil adhered thereto by means of epoxy glue, for example. The thickness of the heavy metal coatings is as described above.

The length of Tm core 44 may generally be in the range of 1.0 to 5.0 mm, preferably about 5 mm, and its diameter may generally be in the range of 0.6 to 1.0 mm, preferably about 0.6 mm. Tube 42 may be manufactured from a type of inert metal, such as, but not limited to, titanium or stainless steel, preferably from titanium. The length of tube 42 may generally be in the range of 2.5 to 8.0 mm, preferably about 5.0 mm, its outer diameter may generally in range of 0.8 to 1.2 mm, preferably about 0.8 mm, and its wall thickness may generally be in the range of 0.05 to 0.1 mm, preferably about 0.1 mm.

After placing the Tm core 44 in tube 42, one of the openings of tube 42 is sealed by a plug 45, said plug 45 is advanced into tube 42 until it abuts Tm core 44. Plug 45 is preferably made from one of the materials indicated above for tube 42, preferably from titanium, its length being about 0.5 mm, and its diameter is adapted to snugly fit into tube 42. After sealing tube 42 with plug 45 and following welding, the radiation source may be activated in a nuclear reactor by means of a neutron flux in the order of 10¹⁴-10¹⁵ n/cm² s. For considerations of safety, the tube is delivered within a quartz ampoule to the nuclear reactor.

A guiding wire 11 is then attached to form part of the device, preferably by means of welding. Alternatively, glue 47 (e.g., epoxy), is placed in the opening of tube 42, and thereafter wire 11 is pressed thereinto until its distal end abuts core 44. This embodiment is useful in implementations in which the different members of the radiation source can not be attached by means of welding.

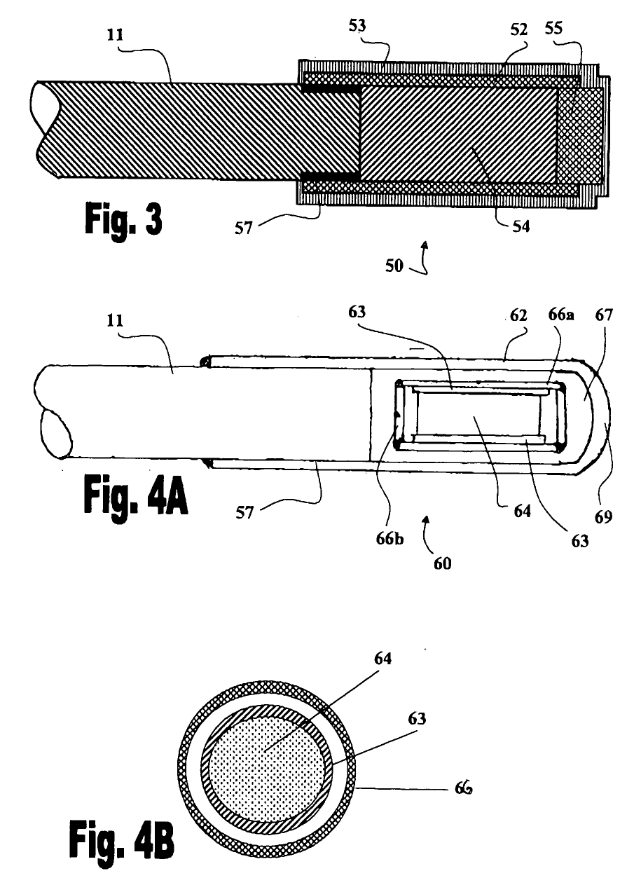

FIG. 3 shows a longitudinal-section view of yet another preferred embodiment of a device 50 of the invention, wherein the heavy metal coating 53 is applied externally. In principle, the construction of radiation source 50 is substantially similar to the construction of device 40 shown in FIG. 2. In this embodiment, however, the Tm core 54 is placed in a tube 52 without the heavy metal coating. One end opening of tube 52 is similarly sealed by a plug 55 which is snugly fitted thereinto until it abuts Tm core 54 and then welded, and after activation, the other end opening of tube 52 is sealed by means of glue 57 applied thereinside and by the distal end portion of wire 11, which is introduced thereinto until its distal tip abuts Tm core 54.

Tm core 54 is preferably cylindrical in shape, its diameter may generally be in the range of 0.6 to 1.0 mm, preferably about 0.6 mm, and its length may generally be in the range of 1.0 to 5.0 mm, preferably about 5 mm. After sealing the end openings of tube 52, tube 52 is covered by one or more heavy metal layers 53 (e.g., Au, Pt or Ce). Heavy metal layers 53 may be applied by means of conventional metal coating techniques, as described above, or alternatively, by wrapping the titanium surface by a foil made of said heavy metal material, and possibly adhering it thereto by means of welding or epoxy glue, for example. Heavy metal layers 53 are preferably made from gold, and their entire length may generally be in the range of 2.5 to 8.0 mm, preferably about 5.0 mm. Heavy metal layers 53 are preferably applied over the entire length of tube 52, namely the entire surface of the tube and the outer surfaces of plug 55 are covered by said layers 53 i.e., the front face of the radiation source is also covered by the heavy metal layers. In this way the intensity of the beta rays emitted from the radiating core is substantially reduced, or stopped, in all directions.

FIG. 4 illustrates a brachytherapy device used for sealing the capsule of the present invention. The device comprises a sealed, elongated tubular body having a distal portion and a proximal portion, wherein the capsule according to the invention is placed in said distal portion of said tubular body and is affixed to said position by means of a rod extending from said proximal portion of said tubular body to said capsule.

More specifically, FIG. 4 shows a longitudinal section view of said device, wherein the capsule is positioned in its distal portion 67. Tm core 64 is encased in an internal enclosure 66 (66a, 66b), which defines the capsule walls. The capsule is prepared as follows. Tm core 64 is wrapped, or coated, by one or more gold, platinum or cerium layers 63 according to the techniques described before. The Tm core 64 with its coating 63 is then placed in a cylindrical enclosure 66, said enclosure may be constructed from a tube 66a made from titanium, for example. After placing the coated Tm core inside the tube 66a its side openings are sealed by means of corresponding plugs or lids 66b, made from the same material of tube 66a, and welding the same thereover or thereinside, to form a capsule according to the present invention.

The thickness of coating layers 63 may generally be in the range of 0.01 to 0.15 mm, preferably about 0.1 mm. The length of Tm core 64 may generally be in the range of 1.0 to 5.0 mm, and its diameter may generally be in the range of 0.5 to 1.0 mm, preferably about 0.6 mm.

The titanium capsule 66 may be then placed in a cylindrical tube 57. Tube 57 may be made of an inert metal, preferably of stainless steel, its length may generally be in the range of 10 to 200 mm, preferably about 100 mm, its outer diameter may generally be in the range of 1.0 to 1.3 mm, preferably about 1.2 mm, and its wall thickness may generally be in the range of 0-05 to 0-1 mm, preferably about 0.1 mm.

The distal end opening of cylindrical tube 57 is sealed by a weld 69 applied thereover such that a distal taper is assumed, and its proximal end is sealed by means of a rod 11 which extends through tube 57 from its proximal end and is contiguous with the titanium capsule, and welding the same therein (rod 11 is welded to tube 57).

In this preferred embodiment of the invention it is assured that the radiating core is effectively sealed inside the radiation source, preferably by means welding, and is safely secured to its desired position in the device.

It is noted that the half-life of ¹⁷⁰Tm is 128 days, which is substantially longer than the half-life of ¹⁹²Ir (74 days). Therefore, the thulium-based source according to the present invention can serve for treating many patients during a long period before it has to be replaced or even reactivated. Since the production of ¹⁷⁰Tm is done by simple neutron activation, any source, which decays below the desired activity, can be reactivated in a neutron flux such that there is no need for a new radiation source whenever the activity of the radiation source substantially decays. The production simplicity of the ¹⁷⁰Tm radiation source is a major advantage of the radiation source of the invention.

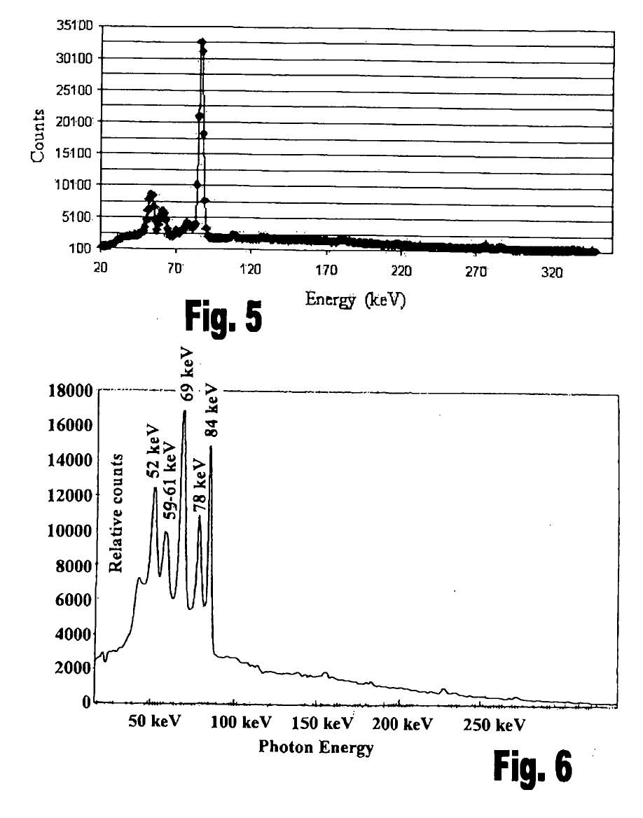

The radiation sources described above may be used in the treatment of both small and large treatment volumes (namely, tumors). The range of the 970 keV beta rays, which comprises 76% of the beta rays emitted from the ¹⁷⁰Tm, is distributed in the irradiated tissue within a radius of about 2 mm from core 14. However, the presence of a metal (gold) coating of thickness of between few atomic layers (e.g., few microns) to about 0.15 mm favorably modifies the radiation emission profile of the source, in cases wherein the beta rays are not wanted or their intensity should be lowered. Specifically, in case of a thick heavy metal layer 13 (e.g., −0.15 mm), all beta radiation of energy of up to 1 MeV is blocked. The intensity of the photon radiation in this case is increased due to the bremssrahlung effect in the heavy metal coating layers 13 in which a part of the beta radiation turns into x-ray photons. The radiation emission profiles of the modified source according to the present invention will now be discussed in more detail with reference to FIGS. 5 to 7. In these figures the abscissa is the energy of the emitted photon and the ordinate is the intensity, recorded by multi-channel analyzer.

The radiation emitted from ¹⁷⁰Tm is composed of beta radiation (electrons) and gamma radiation (photons). 76% of the beta radiation is of energy Emax=968 keV (average energy 237 keV) and 24% of it is of energy 884 keV (average energy 216 keV). FIG. 5 shows the characteristic photon spectrum of ¹⁷⁰Tm (non-coated). As seen in FIG. 5, the photon spectrum has a main peak at 84 keV energy level and another smaller peak, at energy 52 keV. More specifically, there are two photon energies emitted from ¹⁷⁰Tm, the first is composed of gamma rays having a 84 keV energy level emitted from excited 170y atom formed upon the beta decay of ¹⁷⁰Tm, and the second is x-ray at about 52 keV energy level, emitted following the rearrangement of the Y170 electrons, after the interaction of some of the photons and betas emitted from the nucleus, with the atomic electrons. The ratio of gamma to x-ray emission intensity is typically about 3:1.

When the beta rays emitted from the ¹⁷⁰Tm enter the heavy metal layer (e.g., gold) covering the thulium, most of them are stopped by collision with the electrons of the heavy metal atoms. This is the main beta removal effect caused by the heavy metal. A small fraction, less than 3%, undergoes the effect known as braking radiation (Bremsstrahlung in German) due to deceleration of these electrons in the electric field of the heavy metal electrons or the heavy metal nuclei. A portion of the energy of those beta rays is turned into photons of continuous spectrum, mostly in the lower energy range. The fraction of the beta ray turning into photons by braking radiation is proportional to the beta energy times the atomic number of the stopping material. The fraction of the beta intensity turning into photons and the photons energy obtained for each beta ray are shown in table 1 for three possible heavy metals: platinum, gold or cerium. The values in table 1 were calculated for the average energies. The columns marked by the % symbol in table 1 indicate the percent of the beta rays turned into photons in the braking radiation process, for each beta energy. The columns entitled by “Energy” give the photon energy obtained from the beta rays, for each beta energy. The columns entitled by “76%” and “24%” give the actual photon energy obtained, from each beta energy. The last column is the sum of the two, i.e. total photon energy obtained from the beta rays by braking radiation. As mentioned, all the energy values in the table are related to the average beta energy.

TABLE 1—Photon fractions added to the spectrum due to Braking Radiation

| | | | Eₐᵥ = 237 keV | | | Eₐᵥ = 216 keV | | | Total |
|---|---|---|---|---|---|---|---|---|---|
| | Atomic | Density | | Energy | 76% | | Energy | 24% | energy Per |
| Metal | Number | gm/cm³ | % | [keV] | [keV] | % | [keV] | [keV] | Disintegration |
| Pt | 78 | 21.45 | 2.64 | 6.26 | 4.76 | 2.41 | 5.20 | 1.25 | 6.01 keV |
| Au | 79 | 19.3 | 2.68 | 6.35 | 4.83 | 2.44 | 5.27 | 1.26 | 6.09 keV |
| Ce | 58 | 6.75 | 1.96 | 4.65 | 3.53 | 1.79 | 3.87 | 0.93 | 4.46 keV |

When the source (¹⁶⁹Tm coated with one of the heavy metal layers: gold, platinum or cerium in a titanium capsule), is activated by neutrons to get the radioactive isotope ¹⁷⁰Tm, the coating material is activated too and a radioactive isotope is formed. The activation of the titanium is negligible. Pt and Au radioactive isotopes are short lived: T½Pt195=4 days, T½Au198=65 hours, T½Ce141=33 days. The respective activation cross sections are: σPt=10 b, σAu=98.8 b and σCe=0.7 b. The activity of these isotopes is undesired in the radiation source, therefore, after being activated the radiation source should stay in the reactor area for about two weeks cooling period for these isotopes to decay. The two weeks decay time has a minor effect on the ¹⁷⁰Tm activity; it decays to 93% of its activity at the end of the activation. Ce141, the product of ¹⁴⁰Ce neutron activation, emits 36 keV x-rays which is in the desired energy range. It also emits 145 keV gamma rays, but due to the low activation cross-section, 150 times smaller than that of ¹⁶⁹Tm, the amount of these x-rays will be negligible.

The purpose of the radiation emission modifying metal (e.g., Au, Pt or Ce) coating the thulium core is to favorably alter the profile of the radiation generated by the source. Although the metal coating is not radioactive, it emits radiation as a result of absorbing the radiation from the ¹⁷⁰Tm core. The metal coating applied over the radiation source reduces, or stops completely (depends on its thickness), the beta rays emitted from the ¹⁷⁰Tm core, from leaving the radiation source into the diseased site. A part of the beta rays absorbed by the heavy metal coating the Tm turns into photons as braking radiation. An additional photon source is created due to x-ray emitted from the metal coating (this x rays emission is typical to those metals). These x rays energies are in the range of 66-84 keV or 30-84 keV respective to the metal emitting it.

Thus, the metal (Au, Pt or Ce) coating the Tm 170 core of the radiation source changes the radiation emitted therefrom in a number of ways. The effect it has on the beta rays is the most drastic because this thin coating either eliminates the beta rays emission completely or, more preferably, reduces it to a desirable degree, The thicker the coating layer the weaker is the beta intensity.

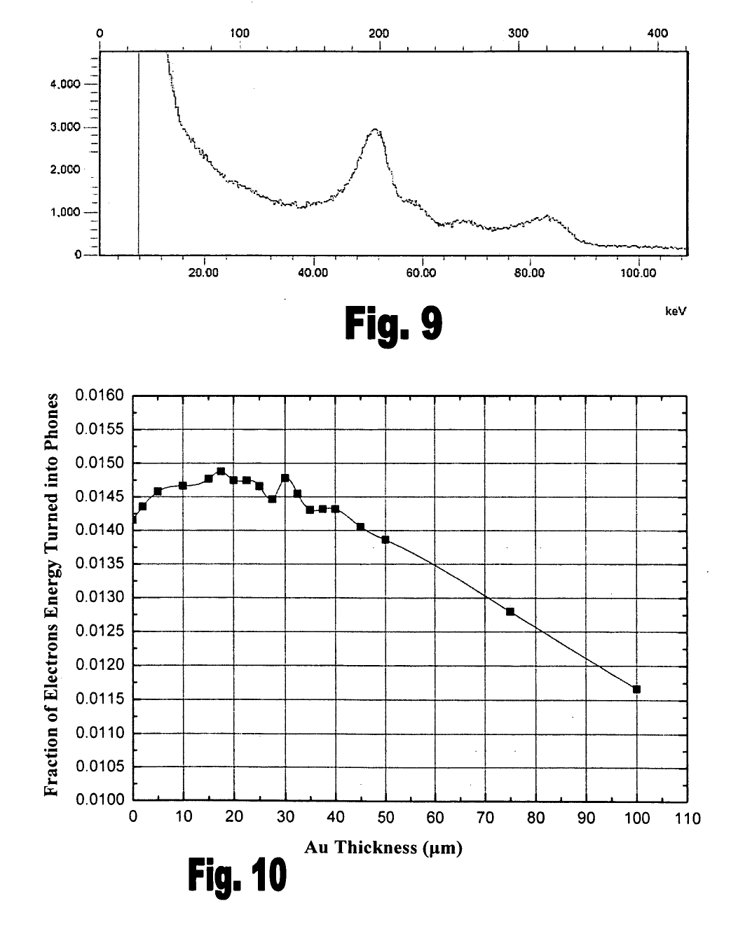

The second effect of the radiation emission modifying metal coating on the radiation emitted from the radiation source is the addition of a continuous x ray spectrum, to the photons emitted from the radiation source. Stopping the beta radiation by the metal coating (Au, Pt or Ce), causes the emission of braking radiation This is a continuous photon spectrum of energy from very low (1-3 keV) to the highest energy of the beta rays-Emax emitted from the ¹⁷⁰Tm. This effect is illustrated in the spectrum shown in FIG. 9. The spectrum was measured with a CZT detector for a ¹⁷⁰Tm source which was coated (in a coaxial fashion) with a 20 micron thick gold layer. Thus, a fraction of the beta radiation generated by thulium-170 is converted by the metal coating into low energy continuous photons. In the graph shown in FIG. 10, the abscissa indicates the thickness of the gold layer which coats the thulium 170, whereas the ordinate relates to the fraction of beta energy converted into photons.

As demonstrated by the graph, a gold layer having thickness of 10-50 (specifically 15-35) microns is especially preferred as far as the conversion of the beta energy into photons energy is concerned.

The third effect of the metal coating on the photon spectrum emitted from the radiation source is the additional peaks in the spectrum, related to x rays emitted from said metal coating. The source of these x rays is the photoelectric effect caused by the 84 keV photons emitted from the Tm 170, namely, the metal coating responds to the 84 keV emission by generating characteristic photons: the main x ray peaks are at energies levels: 67, 69, 78 and 80.1 for gold; 65, 67, 75.7 and 77.8 for platinum. The main gold peaks are seen in FIG. 6. A cerium coating is capable of responding also to the 52 keV photons emitted from the Tm 170, generating low energy photons in the range between 30 and 40 keV, specifically around 34-35 keV (about 34.3, 34.7, 39.2 and 40.0 keV).

In the case of gold coating, the photoelectric effect caused by the 84 keV photons lowers this peak, as can be seen in the spectrum of FIG. 6. (The ratio between the 84 keV peak and the 52.4 keV peak produced by Tm-170 has been reduced compared to that seen in FIG. 5; according to the modified source of the present invention, the ratio between said peaks is less than 2.5:1). This is the fourth spectrum change due to the radiation emission modifying the metal coating onto the ¹⁷⁰Tm. In the case of cerium coating, for example, 100 micron thick cerium layer, both the 84 keV peak and the peaks around 52 keV will be lowered.

To sum up, the changes in the photon spectrum caused by the heavy metal layer are: additional low energy photons, reduction in the 84 keV peak (and possibly in the 52 keV peak) emitted by ¹⁷⁰Tm, and addition of x rays peaks corresponding to the metal coating the thulium radiation source. The change in the beta spectrum: lowering its intensity or complete removal.

The addition of the low energy photons to the spectrum increases the efficiency of the radiation source at small distance. Although lowering the 84 keV photons peak reduces the number of photons that can reach large distance from the source, this is sufficiently compensated by the addition of the higher energy x rays emitted by the heavy metal and by the higher photons in the braking radiation.

As indicated above, the combination of ¹⁷⁰Tm with a radiation emission modifying metal which is either gold or cerium (or both) affords a radiation source suitable for brachytherapy. The photon that can be generated by cerium (at the expense of the 52 and 84 keV rays of ¹⁷⁰Tm) has energy of about 35 keV, and accordingly, its traveling distance is not more than a few centimeters (such that the combination of thulium and cerium may be especially useful in treating prostate cancer). Photons that can be generated by gold (at the expense of the 84 keV ray of ¹⁷⁰Tm) have energies of about 67-70 keV, and accordingly, their traveling distance is not more than about 30 cm. Calculations were made for the dose distributions around a non-coated ¹⁷⁰Tm source and ¹⁷⁰Tm coated with either a gold layer or cerium layer (of thicknesses of 20 and 100 microns, respectively). The calculations were made for sources implanted in tissue with the results representing the dose in 5 mm³ cubes, in units of cGy/hr per lCi; the calculations were done using the EGS5 Monte Carlo code. The results obtained show that the coated ¹⁷⁰Tm radiation sources of the invention generate appreciable beta and gamma emission, with the concomitantly formed photons (generated by the gold or cerium coating at the expense of the 84 keV ray of ¹⁷⁰Tm) being of lesser energy and hence lesser danger of penetrating healthy tissue beyond the tumor volume.

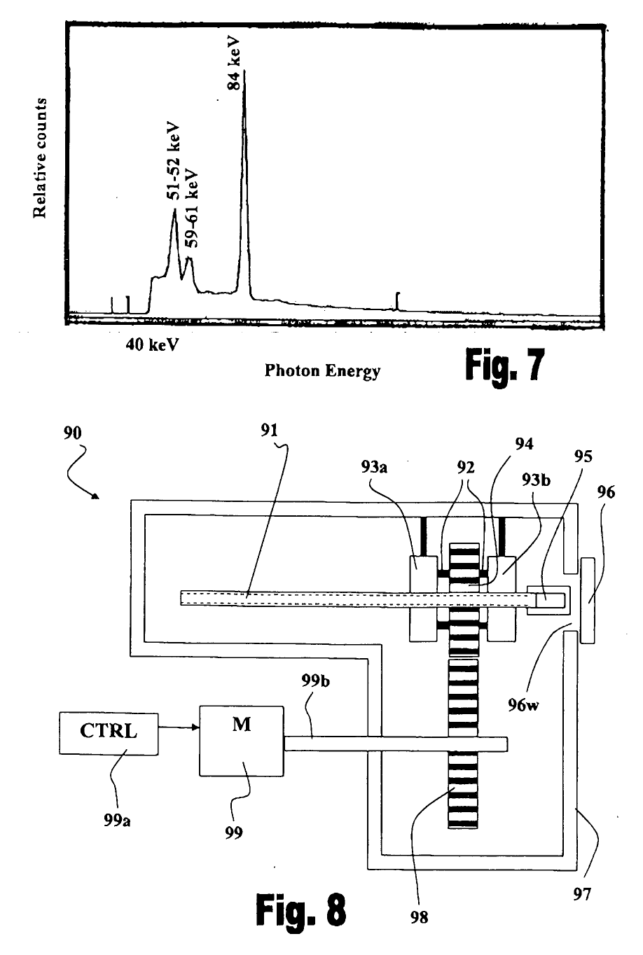

FIG. 7 (comparative) shows the photon spectrum of a ¹⁷⁰Tm radiation source in which the radioactive section was not coated by gold but encased in a titanium capsule having wall thickness of about 0.05 mm. The continuous spectrum beyond the 84 keV peak is due to braking radiation in the titanium.

The radiation source of the invention may be introduced into the body of the treated subject by employing conventional HDR procedures. For example, in use, the doctor inserts a catheter such as a hollow plastic tube into the patient's body. A mechanical computer-controlled delivery system called an afterloader is then used to remotely remove the radioactive source from the safe and move it through the catheter to the tumor site. The remote afterloading technique reduces the radiological risk to medical personnel of exposure to the high-activity source. A suitable procedure is also described in U.S. Pat. No. 6,607,478. Commercial HDR brachytherapy systems are available from Varian and Nucletron.

FIG. 8 schematically illustrates a delivery system 90 designed for remotely advancing a radiation source into a treated subject. The radiation source 95 is stored in a lead box 97 having wall thickness about 1 cm. Lead box 97 houses the delivery mechanism, which is comprised of a stepping motor 99 (external to lead box 97) driven by a controller 99a, a gear 98, a driving nut 94, bearings 93a and 93b, and the threaded rod 91 holding the radiation source 95. The motor 99 turns a gear wheel 9B, turning a nut 94, which in turn drives the rod 91 with the radiation source 95 forward or backward.

Rod 91 is threaded along most of its length and it passes through a threaded passage provided in nut 94. Strainers 92 keep the nut-gear 94 from moving along the rod as it turns. The bearings 93a and 93b are tight enough to keep the rod 91 from turning when the nut 94 is turning, but not too tight so that the rod can move back and forth.

When the radiation source is moved forward into the patient or back into the shielded box, corresponding control signals received from controller 99a (electronic pulses received from a pulse generator or a suitable control logic may be employed) activates stepping motor 99 to rotate the nut 94 the respective number of turns for placing the radiation source 95 in the exact position needed.

The thickness of lead box is preferably in the range of 0.5-1.0 cm, such that no radiation escapes therefrom. A window 96w is provided in one of the walls of lead box 97 through which the radiation source 95 is passed into the patient's body. Window 96w is covered with a lead door 96 having wall thickness similar to that of lead box 97. Lead door 96 is removed before the radiation source 95 is sent out of lead box 97.

The rod 91 holding the ¹⁷⁰Tm source can be rigid so that it moves back and forth along a straight line only. Alternatively, the rod 91 holding the ¹⁷⁰Tm source is made flexible such that it can be guided through curved passages to locations within the patient body which are not exactly in front of the window 96w.

### EXAMPLES

### Example 1

### Preparation of a Titanium Capsule Comprising Thulium 170 and Gold

Thulium 169 wire of 0.6 mm diameter was cut to obtain a section having a desired length (in the range between 1.0 mm and 5.0 mm). The thulium piece was then wrapped with a gold foil having thickness of about 0.1 mm (Goodfellow, England).

One end of a titanium tube (0.8 mm outer diameter, 0.7 inner diameter) was closed with a titanium plug. The plug is made of a titanium wire of diameter 0.7 mm (the inner diameter of the tube). A slice of 0.5 mm long of this wire was plugged into the titanium tube on one end and welded with a laser beam. The length of the titanium tube used depends on the length of the thulium piece+0.5 mm for a plug insertion on each end.

The gold coated thulium was inserted into the titanium tube through the open end. The second end of the tube was also plugged and sealed by welding. The titanium tube is now ready to be activated in order to convert the ¹⁶⁹Tm into ¹⁷⁰Tm (the high flux reactor in Peten, the Netherlands). The Activation time and flux level, which determine the source activity, were described hereinabove.

### Example 2

### Preparation of a Brachytherapy Device, Comprising Thulium 170 and Gold within a Titanium Shell Attached to a Guidewire (the Embodiment Shown in FIG. 2)

A thulium wire of 0.6 mm diameter was cut to obtain a section having a desired length (in the range between 1.0 mm and 5.0 mm). The thulium piece was then wrapped with a gold foil having thickness of 0.1 mm (Goodfellow, England).

One end of a titanium tube (0.8 mm outer diameter, 0.7 inner diameter) was plugged with a titanium plug. The plug is made of a titanium wire of diameter 0.7 mm (the inner diameter of the tube). A slice of 0.5 mm long of this wire was plugged into the titanium tube on one end and welded with a laser beam. The length of the titanium tube used depends on the length of the thulium piece+0.5 mm for a plug insertion on one end and ² mm (or more) for inserting the flexible steel cable on the other end.

The gold coated thulium section was inserted into the titanium tube through the open end. The assembly was shipped to a nuclear reactor for neutron activation (the high flux reactor in Peten, the Netherlands). The Activation time and flux level, which determine the source activity, were described hereinabove.

After neutron activation, the thulium is radioactive and should be handled with the required care, and safety measures should be taken. The preparation of the source was completed in a hot cell or behind sufficient shielding. The steel wire was inserted into the open end of the titanium tube, using an epoxy glue and mechanical pressure to ensure a safe connection.

### Example 3

### Preparation of Brachytherapy Device Comprising the Capsule of the Present Invention (the Embodiment Shown in FIG. 4)

A needle commonly applied in brachytherapy to implant radioactive seeds into patients was used to house the titanium capsule of Example 1, according to the following procedure (these needles have an inner diameter of 1.1 mm, outer diameter of 1.3 mm and are 200 mm long). The sharp end of the needle is removed and this end of the needle is carefully sealed by welding with copper or silver (care must be taken to keep the outer diameter of the needle as it is when seal-welding the end, otherwise it might not fit into the catheter used for delivering the source into the patient). The capsule is placed inside the needle through the open end. It is pushed all the way to the distal, sealed end using the inner part of the needle. This is a steel rod 1.0 mm diameter, 200 mm long, which is used to retain the capsule in place inside the needle. The other end of the needle, with the steel rod inside, is now welded to obtain a sealed source on both ends.

### Example 4

### Preparation of a Titanium Capsule Comprising Thulium-170 Plated with Gold

This example illustrates the preparation of the capsule according to the invention, wherein a thulium wire was plated with gold, applying a spattering technique. E5100 Polaron system was used for this purpose.

A gold foil, less than 1.0 mm thick on aluminum base was used as the gold source. The gold foil has a shape of a flat ring 10 cm diameter, 1 cm thick. This gold ring and the thulium cylinder (thulium wire 0.6 mm diameter, 5 mm long) to be plated were placed in a vacuum chamber. The air was pumped out and argon gas was let into the chamber to a pressure of 0.1 to 0.2 Torr. The argon is kept flowing into the chamber during the gold plating to keep the pressure constant. A voltage of the order of 1-2 kV was applied between the gold electrode and the thulium wire pieces. The distance between the gold electrode and the thulium wire was about 50 mm. Ion current of about 20 mA flowed between the gold electrode and the thulium wire, causing gold spattering from the gold electrode. Under such conditions, every five minutes the thickness of the gold layer increases by 75 Å. The growth of the gold coating was allowed to continue for 60 minutes. The gold-coated thulium is inserted into a titanium capsule and sealed. The sealed capsule is delivered to a nuclear reactor to be activated as described hereinabove.

### Example 5

The following example describes an experiment which demonstrates the efficiency of the Tm-170+Au beta-gamma radiation source of the invention.

A Tm-170 radiation source for HDR having a gold coating was produced by irradiation of 13 mg natural thulium (¹⁶⁹Tm) rod, 5 mm long, 0.6 mm diameter, at the nuclear reactor in Peten, The Netherlands. The irradiation lasted 21 days at a neutron flux of 2.5×10¹⁴ n/cm²·sec. The activity of the ¹⁷⁰Tm obtained was 5.4 curie. The rod was contained in a titanium capsule of 0.8 mm o.d., 0.7 mm i.d., 7 mm long. For the production of a usable source the capsule was sealed in a stainless steel needle, 20 cm long. 1.3 mm o.d, welded on both ends. The needle was then wrapped, at the source (distal) end, with a 0.1 mm thick gold foil. The Tm-170 source was kept in place inside the needle by a stainless steel rod inserted into the needle before welding the proximal end.

17 tumor bearing white mice were irradiated using the source described above. The mice were divided into two groups, 9 were irradiated with the gold foil wrapping the source, and 8 without the gold foil. Each of these two groups was divided into 3 irradiating time groups: 5 minutes, 10 minutes and 15 minutes. The mice were placed in a lead box of 3 mm wall thickness to protect their body from the radiation. A hole was made in the box through which the tumor was pushed out, for irradiation. A 2 mm o.d, 1.5 mm i.d, 15 cm long needle was inserted into the tumor to serve as a guide for the source. The experiment was done behind a thick lead-glass shielding with lead shielding on all other sides. The source was pushed into the guiding needle and was kept at the tumor for time length as mentioned above.

The tumor volume was measured every day before and after the treatment. In all mice the tumor growth stopped for days following the irradiation, or the tumor size was reduced. In order to eliminate the tumor completely, a second irradiation was needed about a week after the first one.

The experiment above shows that ¹⁷⁰Tm in combination with a gold coating is equally effective in treating cancer in comparison with non-coated Tm-170, despite the reduced beta emission. The gold coated Tm-170 has the advantage of having more photons absorbed in the tumor and is hence likely to be particularly useful in treating large tumors.

All of the abovementioned parameters are given by way of example only, and may be changed in accordance with the differing requirements of the various embodiments of the present invention. Thus, the abovementioned parameters should not be construed as limiting the scope of the present invention in any way. In addition, it is to be appreciated that the different cylindrical members, rods, wires, tubes, and other members, described hereinabove may be constructed in different shapes (e.g. having oval, square etc. form in plan view) and sizes differing from those exemplified in the preceding description.

1) A capsule for high dose rate brachytherapy, wherein the capsule comprises within its interior space thulium 170 (¹⁷⁰Tm), and further comprises at least one layer of a radiation emission modifying metal, wherein said at least one layer is provided internally within the capsule, or on the outer surface thereof, or both.

2) A capsule according to claim 1, wherein the radiation emission profile of the radiation source is characterized in that:
(i) the level of the beta radiation emitted is reduced in comparison with that generated by ¹⁷⁰Tm in the absence of the radiation emission modifying metal;
wherein said radiation emission profile being further characterized in one or more of the following:
(ii) the photon emission spectrum of said source exhibits one or more peaks in the region between 30 keV and 84 keV energy levels, or between 65 and 84 keV, indicative of photons emitted by the layer(s) of the radiation emission modifying metal;
(iii) the photon emission spectrum of said source further comprises, in addition to discrete peaks, a continuous region indicative of the emission of braking energy associated with the layer(s) of the radiation emission modifying metal;
(iv) the photon emission spectrum of said source displays a lower ratio between the intensities of the characteristic peaks at 84 keV and 52 keV energy levels, relative to the ratio observed between said peaks in the spectrum of thulium 170 in the absence of the radiation emission modifying metal.

3) A capsule according to claim 2, wherein the radiation emission profile of the radiation source is characterized in that the photon emission spectrum of said source exhibits one or more peaks in the region between 65 keV and 84 keV energy levels indicative of photons emitted by the layer(s) of the radiation emission modifying metal.

4) A capsule according to claim 2, wherein the radiation emission modifying metal is provided as a gold coating, a platinum coating or a cerium coating, or combination thereof.

5) A capsule according to claim 4, wherein the capsule is made of titanium.

6) A capsule according to claim 4, wherein the thulium-170 is in the form of a cylindrical body and a gold coating is provided on the surface of said thulium-170, such that said coating is interposed between said thulium 170 and the walls of the capsule.

7) A high dose rate brachytherapy device which comprises as a radiation source thulium-170 and at least one layer of a radiation emission modifying metal surrounding said thulium-170, wherein the photon emission spectrum of the source exhibits one or more peaks in the region between 30 keV and 84 keV energy levels indicative of photons emitted by the layer of the radiation emission modifying metal.

8) A high dose rate brachytherapy device according to claim 7, wherein the photon emission spectrum of the source exhibits one or more peaks in the region between 65 keV and 84 keV energy levels indicative of photons emitted by the layer of the radiation emission modifying metal.

9) A high dose rate brachytherapy device according to claim 7, wherein the photon emission spectrum of the radiation source exhibits a continuous spectrum of energy, indicative of the emission of braking energy associated with the layer(s) of the radiation emission modifying metal.

10) A high dose rate brachytherapy device according to claim 7, wherein the radiation emission modifying metal is selected from the group consisting of gold, platinum, cerium and a mixture thereof.

11) A high dose rate brachytherapy device according to claim 7, comprising
a) a thulium-170 radioactive section attached to one end of a guiding wire, said radioactive section and a portion of said guiding wire being placed within the interior of a shell and affixed to the walls thereof, with the proximal end of the guiding wire extending beyond said shell; and
b) a radiation emission modifying metal, provided either internally within the shell or on the outer surface thereof.

12) A high dose rate brachytherapy device according to claim 11, wherein the radiation emission modifying metal is provided in the form of a gold coating, a platinum coating or cerium coating onto the radioactive section, such that said coating is interposed between said radioactive section and the shell.

13) A high dose rate brachytherapy device according to claim 11, wherein the radiation emission profile of the source placed therein is characterized in that:
(i) the level of beta radiation emitted therefrom is reduced in comparison with that generated by Tm-170;
(ii) the photon emission spectrum of said source displays a lower ratio between the intensities of the characteristic peaks at 84 keV and 52 keV energy levels, relative to the ratio observed in the corresponding spectrum of Tm-170;
(iii) the photon emission spectrum of said source exhibits one or more peaks in the region between 65 keV and 84 keV energy levels indicative of photons emitted by the layer(s) of the radiation emission modifying metal;
(iv) the photon emission spectrum of said source comprises in addition to the discrete peaks also a continuous region indicative of the emission of braking energy associated with the layer(s) of the radiation emission modifying metal.

14) A capsule according to claim 1, wherein the radiation source has an activity in the range of 3 to 70 curie.

15) A brachytherapy device according to claim 7, wherein the radiation source has an activity in the range of 3 to 70 curie.

16) A high dose rate brachytherapy method, which comprises placing proximate to the diseased site one or more radiation sources, each comprising thulium-170 provided with a coating of a radiation emission modifying metal, wherein the activity of said radiation source is not less than 3 curie, and delivering radiation dose to said diseased site.

17) A method according to claim 16, wherein the radiation emission modifying metal is selected from the group consisting of gold, platinum, cerium and mixtures thereof.

18) A method according to claim 17, wherein the radiation emission modifying metal is gold.
